(19) United States
(12) Patent Application Publication
(10) Pub. No.: US 2026/0162843 A1
(43) Pub. Date: Jun. 11, 2026

(54) METHOD FOR PRODUCING MEDICAL GRADE LEAD-212

(71) Applicant: ORANO MED MANUFACTURING, Chatillon (FR)

(72) Inventors: Rémy DUREAU, Saint Gence (FR); Mélissa FLEURAT, Saint Jouvent (FR); Alexandre MALTERRE, Limoges (FR); Julien TORGUE, Gaithersburg, MD (US)

(21) Appl. No.: 19/408,945

(22) Filed: Dec. 4, 2025

(30) Foreign Application Priority Data
Dec. 5, 2024 (FR) FR2413470

(51) Int. Cl. G21G 4/08; A61K 51/12; G21G 1/00

(52) U.S. Cl. CPC G21G 4/08; A61K 51/121; G21G 1/001; G21G 2001/0094

(57) ABSTRACT

The application relates to a method for producing medical grade lead-212, including the steps of:
a) producing lead-212 by radioactive decay of radium-224 in at least one first chromatography column containing a first stationary phase to which radium-224 is attached;
b) eluting thus produced lead-212 from the first stationary phase;
c) loading the eluate thus obtained into a second chromatography column containing a second stationary phase to attach the lead-212 to the second stationary phase;
d) performing wash(es) of the second stationary phase; and then
e) eluting lead-212 from the second stationary phase;
   and wherein:
step b) includes circulating in said at least one first chromatography column an aqueous solution A1 including from 0.8 mol/L to 1.6 mol/L chloride, optionally with at most 200 mmol/L HCl; and
step d) includes circulating in the second chromatography column an aqueous solution A2 including from 0.01 mol/L to 1 mol/L chloride.

### CROSS-REFERENCE TO RELATED APPLICATIONS

This application claims the priority of French Patent Application No. FR2413470, filed May 5, 2024, which is incorporated herein by reference in its entirety.

### DESCRIPTION

### Technical Field

The invention is directed to the field of the production of radioactive isotopes, also called radioisotopes.

More specifically, it is directed to a method that makes it possible to produce lead-212 with a very high degree of radiological purity, making it fully suitable for medical use.

This method is therefore likely to find applications in the manufacture of lead-212-based radiopharmaceuticals, useful in nuclear medicine and, in particular, in alpha radiation therapy targeting cancer treatment.

### State of Prior Art

Lead-212 is a rare radioactive lead isotope, which has been the subject of promising research for several years, particularly for treatment with targeted alpha radiation therapy, also called targeted alpha therapy, for cancers and, in particular, for pancreatic, ovarian, colon, breast and prostate cancers.

Lead-212 is also one of the radioisotopes that has been shown to be of interest in medical imaging, in particular for performing examinations by single photon emission computed tomography coupled with a scanner.

In both cases, the use of lead-212 implies that it is injected into the patient in the form of a radiopharmaceutical, that is a product in which it is bound, typically via a chelating agent, to a molecule capable of very specifically targeting the cells desired to be destroyed (if it is targeted alpha therapy) or observed (if it is medical imaging), such as a peptide.

To achieve this, lead-212 must meet extremely stringent quality requirements and, in particular, radiological purity, this should ideally be at least 99.95%.

In this respect, it is set out that the radiological purity of a radioisotope such as lead-212 is understood to mean the purity that this radioisotope has with respect to the radioisotopes from which it is derived by radioactive decay (that is its ascendants) as well as with respect to other radioisotopes that are not part of its radioactive decay chain, and not the purity that this radioisotope exhibits with respect to the radioisotopes to which it gives rise by its own radioactive decay (that is its descendants).

As illustrated in FIG. 1 attached in the appendix, which represents the radioactive decay, also known as disintegration, chain of thorium-232, lead-212 belongs to the radioactive family of thorium-232 of which it is a daughter product. It is also a daughter product of thorium-228 and radium-224, which are situated, in this chain, between thorium-232 and lead-212.

To produce medical grade lead-212, that is meeting the aforementioned radiological purity requirements, methods have been provided in international applications PCT WO-A-2013/174949 and WO-A-2017/093069, hereinafter references [1] and [2], which schematically comprise:
producing lead-212 by radioactive decay of radium-224 in a generator which comprises a stationary phase to which radium-224 is attached and which will be more simply referred to as the Ra-224/Pb-212 generator in the following;
eluting lead-212 thus produced from the stationary phase of the Ra-224/Pb-212 generator, then
purifying thus eluted lead-212 by liquid chromatography.

These references also describe devices specially designed for the automated implementation, in a closed system, of these methods.

It is found that the references [1] and [2] both provide for the elution of lead-212 of the stationary phase of the Ra-224/Pb-212 generator with an aqueous solution comprising from 1.5 mol/L to 2.5 mol/L strong acid of the hydrochloric acid or nitric acid type, the acid used in the examples of these references being 2 mol/L hydrochloric acid. This elution results in obtaining of a strongly acidic eluate which is then loaded into the chromatography column serving to purify lead-212.

However, the use of hydrochloric acid at concentrations recommended in references [1] and [2] and, in particular, 2M hydrochloric acid poses corrosion problems for equipment likely to be used for the production of lead-212, such as automated elution apparatuses including some components (valve bodies, fittings or bearings for example) that are sensitive to acids as well as neighboring steel surfaces such as those of glove boxes or shielded chains with the result of a high maintenance rate.

Furthermore, in reference [2], it is contemplated that the method may comprise, upstream of the production of lead-212 in the Ra-224/Pb-212 generator, a step aimed at producing the radium-224 itself by radioactive decay of thorium-228 in a generator which comprises a stationary phase to which this thorium is attached and which will be referred to more simply as the Th-228/Ra-224 generator hereinafter. It is set out that, in this case, elution of radium-224 from this stationary phase with a view to its recovery is carried out with an acidic aqueous solution such as a hydrochloric acid solution. The hydrochloric acid concentration of this elution solution is not mentioned but it can be deduced from the reference [2] that this concentration is between 1 mol/L and 3 mol/L since the eluate comprising radium-224, which is then used to attach this radium on the stationary phase of the Ra-224/Pb-212 generator, has a hydrochloric acid concentration between 1 mol/L and 3 mol/L and preferably 2 mol/L. The use of such an elution solution therefore poses the same corrosion problems as those previously discussed. In addition, the retention curve of thorium-228 by the DGA resin (Triskem International) whose use is encouraged in reference [2], as the stationary phase of the Th-228/Ra-224 generator, shows a very significant slope in 1M to 3M hydrochloric medium. As a result, the least approximation in the molarity of this acid exposes to a high risk of thorium-228 leaks and/or decreased generator lifetime.

In view of the foregoing, the inventors have set the objective of providing a lead-212 production method which, while exhibiting the same performance as the methods described in references [1] and [2], in particular in terms of yield and quality of the lead-212 produced, is free of the drawbacks discussed hereinabove.

They have also set the objective of improving the elution performance of lead-212 during its purification by liquid chromatography and, more specifically, of achieving a reduction in the volume of elution solution required to elute all the available activity of lead-212.

They have further set the objective that this method can be implemented in an automated manner, for example by means of one of the devices described in references [1] and [2].

### DISCLOSURE OF THE INVENTION

These objectives are met by the invention which provides a method for producing medical grade lead-212, comprising at least the steps of:
a) producing lead-212 in at least one Ra-224/Pb-212 generator, that is by radioactive decay of radium-224 present in at least one first chromatography column containing a first stationary phase to which radium-224 is attached;
b) eluting thus produced lead-212 from the first stationary phase to obtain an eluate comprising unpurified lead-212;
c) loading the eluate thus obtained into a second chromatography column containing a second stationary phase to attach the lead-212 present in the eluate to the second stationary phase;
d) performing wash(es) of the second stationary phase to remove radioactive impurities likely to be retained by the second stationary phase without removing lead-212; and
e) eluting lead-212 from the second stationary phase, whereby medical grade lead-212 is obtained in aqueous solution;
   and which is characterized in that:
step b) comprises circulating in said at least one first chromatography column an aqueous solution A1 comprising from 0.8 mol/L to 1.6 mol/L chloride, alone or in mixture with at most 200 mmol/L hydrochloric acid; and
step d) comprises circulating in the second chromatography column an aqueous solution A2 comprising from 0.01 mol/L to 1 mol/L chloride.

Thus, according to the invention, the aqueous solution, which is used in step b) to elute lead-212 from the stationary phase of the Ra-224/Pb-212 generator(s), is a solution which comprises a chloride and in which hydrochloric acid may be present but at a concentration infinitely lower than that encouraged in references [1] and [2]. Consequently, the same applies to the eluate that is loaded into the second chromatography column in step c).

Furthermore, the wash(es) of the stationary phase to which lead-212 is attached—or step d)—is (are) carried out with an aqueous solution comprising a chloride, and not with an aqueous hydrochloric acid solution as described in the examples of references [1] and [2].

The risks of corrosion of the equipment and neighboring surfaces are thus eliminated or at least drastically reduced, with the consequence of a reduction in the maintenance rate and, therefore, in the production costs of lead-212, without affecting the level of radiological purity obtained.

In accordance with the invention, the first and second stationary phases are preferably of the same type as those used in references [1] and [2] to produce lead-212 in the Ra-224/Pb-212 generator and then purify this lead respectively, namely:
the first stationary phase is preferably a cation exchange resin that retains radium, regardless of the isotope thereof, but does not retain lead in the presence of chloride ions, regardless of the isotope thereof, such as a resin consisting of particles of an organic polymer, such as poly(styrene-co-divinylbenzene), to which sulfonic groups, SO₃H, are grafted, of the type available from the company Bio-Rad under the trade name AG™ MP-50, while
the second stationary phase is preferably an extraction resin which consists of particles of an inert carrier impregnated with an ether-crown, such as a dicyclohexano-18-crown-6 or a dibenzo-18-crown-6 of which the cyclohexyl or benzyl groups are substituted with one or more straight or branched-chain C₁-C₁₂ alkyl groups; thus, the second stationary phase may in particular be a resin whose inert carrier is impregnated with 4,4′(5′)-di-tert-butylcyclohexane-18-crown-6 in isodecanol solution, such as that available from the company Triskem International under the trade name Resin PB.

The chloride present in the aqueous solutions A1 and A2 may be selected from many salts comprising at least one chloride anion. In particular, it may be a chloride of a metal and, in particular, a chloride of an alkali metal, such as sodium or potassium chloride, or of an alkaline earth metal, such as calcium or magnesium chloride, or even ammonium chloride. Of these salts, preference is given to magnesium chloride.

In accordance with the invention, the aqueous solution A1 preferably comprises from 0.8 mol/L to 1.2 mol/L and more preferably 1.0 mol/L magnesium chloride, alone or in mixture with at most 50 mmol/L hydrochloric acid, this acid, if present, being preferably at a concentration of 1 mmol/L.

The aqueous solution A2 preferably comprises from 0.1 mol/L to 1 mol/L and, more preferably, 1 mol/L magnesium chloride.

Given that, like any chromatography column, the second column has two opposite ends, respectively called column head and column tail, step d) comprises circulating a first volume of aqueous solution A2 from the column head to the column tail, then circulating a second volume of aqueous solution A2, identical to or different from the first volume, from the column tail to the column head.

Step e) in turn comprises circulating in the second chromatography column an aqueous solution A3, which advantageously has a pH between 5 and 9 and preferably comprises one or more complexing or chelating agents—the two terms being considered synonymous herein—and/or antioxidant agents.

The complexing or chelating agent(s) may especially be selected from:
ammonium acetate preferably used in a concentration ranging from 0.15 mol/L to 1 mol/L;
citric acid and salts thereof such as citrates of an alkali metal (such as monosodium citrate, disodium citrate or trisodium citrate), citrates of an alkaline earth metal (such as monocalcium citrate, dicalcium citrate or tricalcium citrate) or ammonium citrates such as monobasic ammonium citrate, dibasic ammonium citrate or tribasic ammonium citrate, which are preferably used at a concentration ranging from 10 mmol/L to 200 mmol/L, preference being given to citric acid; and
chelators that are typically used in the preparation of products intended for nuclear medicine and, in particular, cyclene derivatives, that is 1,4,7,10-tetraazacyclododecane, such as DOTA (or 1,4,7,10-tetraazacyclodecane-1,4,7,10-tetraacetic acid) or DOTAM (or 1,4,7,10-tetraazacyclodecane-1,4,7,10-tetraacetic amide), which then saves valuable time in the manufacture of lead-212-based radiopharmaceuticals given that the half-life thereof is only 10.6 hours; if such chelators are used, then they are preferably at 0.2 μmol/L to 200 μmol/L.

The antioxidant agent(s), for their part, may in particular be selected from ascorbic acid and citric acid, which are preferably used at a concentration ranging from 10 mmol/to 200 mmol/L.

Advantageously, the aqueous solution A3 comprises ammonium acetate and citric acid (which acts as both a complexing agent and an antioxidant agent) and/or DOTAM. Thus, for example, the aqueous solution A3 may comprise 0.4 mol/L ammonium acetate and 75 mmol/L citric acid or 0.4 mol/L ammonium acetate, 75 mmol/L citric acid and 2 μmol/L DOTAM.

Either way, the aqueous solution A3 is preferably circulated in the second chromatography column from the column tail to the column head.

Advantageously, the method is implemented using m Ra-224/Pb-212 generators, that is m first chromatography columns each containing a first stationary phase to which radium-224 is attached, m being an integer at least equal to 2, typically between 2 and 4, and a single second chromatography column.

In this case, the first m chromatography columns may be disposed in parallel, in which case:
steps a) and b) are performed in each of the first m chromatography columns, whereby m eluates comprising unpurified lead-212 which are collected separately or together to form a mixture of the m eluates are obtained;
step c) is performed by loading the m eluates or the mixture of the m eluates thus obtained into the second chromatography column containing the second stationary phase;
step d) of washing the second stationary phase is performed; and
step e) of eluting lead-212 from the second stationary phase is performed.

Thus, by loading these m eluates (separately or in the form of a mixture) into the second chromatography column, it is possible to increase the amount of unpurified lead-212 that attaches to the second stationary phase and, hence, to concentrate lead-212 in the aqueous solution derived from the elution provided in step e).

Alternatively, the first m chromatography columns can be connected in series, in which case:
steps a) and b) are performed in each of the first m chromatography columns, step b) being carried out by circulating the aqueous solution A1 successively in said first m chromatography columns, whereby an eluate comprising unpurified lead-212 is obtained upon leaving the m^{th} of these columns;
step c) is performed by loading the eluate thus obtained into the second chromatography column containing the second stationary phase;
step d) of washing the second stationary phase is performed; and
step e) of eluting lead-212 from the second stationary phase is performed.

Thus, with this alternative, it proved possible to elute lead-212 from m Ra-224/Pb-212 generators using a substantially lower volume of aqueous solution A1 than that which would be necessary to elute the same amount of lead-212 from m Ra-224/Pb-212 generators having the same lead-212 load but which would not be connected in series with, ultimately, a saving of reagents, a concentration of unpurified lead-212 in the only eluate obtained upon leaving the m^{th} Ra-224/Pb-212 generator and, thereby, better attachment of this lead to the stationary phase of the second chromatography column (since present in a lower volume) and a concentration of lead-212 in the aqueous solution derived from the elution provided in step e).

Regardless of the alternative, two or three first chromatography columns are preferably used, that is m=2 or 3.

In accordance with the invention, the method preferably further comprises, before step a), the steps of:
i) producing radium-224 in at least one Th-228/Ra-224 generator, that is by radioactive decay of thorium-228 in at least one third chromatography column comprising a third stationary phase to which the thorium-228 is attached; and
ii) eluting radium-224 thus produced from the third stationary phase to obtain an eluate comprising radium-224, eluting comprising circulating in said at least one third chromatography column an aqueous solution A0 comprising from 0.4 mol/L to 1 mol/L nitric acid; and then
iii) loading the eluate thus obtained into said at least one first chromatography column to attach radium-224 present in the eluate to the first stationary phase.

In accordance with the invention, the third stationary phase is, preferably, an extraction resin which consists of particles of an organic polymer, such as a polymethacrylate or a poly(styrene-co-divinylbenzene), impregnated with an actinide ligand such as a tetraalkylated diglycolamide (for example, N,N,N′,N′-tetraoctyl-diglycolamide or TODGA), a dialkylphosphoric acid (for example, di(2-ethylhexyl)phosphoric acid or HDEHP) or a trialkylphosphine oxide (for example, trioctylphosphine oxide or TOPO).

Thus, the third stationary phase may especially be a resin whose polymer is impregnated with TODGA, such as that available from the company Triskem International under the trade name Resin DGA Normal.

Preferably, the aqueous solution A0 comprises from 0.5 mol/L to 0.75 mol/L and, more preferably, 0.5 mol/L nitric acid.

Advantageously, n Th-228/Ra-224 generators are used, that is n third chromatography columns each containing a third stationary phase to which thorium-228 is attached, n being an integer at least equal to 2, typically between 2 and 5.

In this case, the n third chromatography columns may be disposed in parallel, in which case:
steps i) and ii) are performed in each of the n third chromatography columns, whereby n eluates comprising radium-224 which are collected separately or together to form a mixture of the n eluates are obtained; and
step iii) is performed by loading the n eluates or the mixture of the n eluates thus obtained into said at least one first chromatography column.

This makes it possible to increase the amount of radium-224 that attaches in step iii) to the stationary phase contained in said at least one first chromatography column and, hence, to increase the amount of lead-212 produced by radioactive decay of radium-224 in step a).

Alternatively, the n third chromatography columns can be connected in series, in which case:
steps i) and ii) are performed in each of the n third chromatography columns, step ii) being performed by circulating the aqueous solution A0 successively in said n third chromatography columns, whereby an eluate comprising radium-224 is obtained upon leaving the n^{th} of these columns; and
step iii) is performed by loading the eluate thus obtained into said at least one first chromatography column.

Here, too, this alternative makes it possible to elute radium-224 from the n Th-228/Ra-224 generators using a volume of aqueous solution A0 that is considerably lower than that which would be necessary to elute the same amount of radium-224 from n Th-228/Ra-224 generators having the same radium-224 load but which would not be connected in series with, ultimately, a saving of reagents, a concentration of radium-224 in the only eluate obtained upon leaving the n^{th} Th-228/Ra-224 generator and, consequently, better attachment of this radium to the stationary phase of said at least one chromatography column or, in other words, said at least one Ra-224/Pb-212 generator.

Regardless of the alternative, two or three third chromatography columns are preferably used, that is n=2 or 3.

Further characteristics and advantages of the method of the invention will appear upon reading the following additional description, which is directed to implementations of this method.

It goes without saying that these implementations are given for illustrative purposes only and are in no way limiting to the object of the invention.

### BRIEF DESCRIPTION OF THE FIGURES

FIG. 1, already commented, represents the radioactive decay chain of thorium-232.

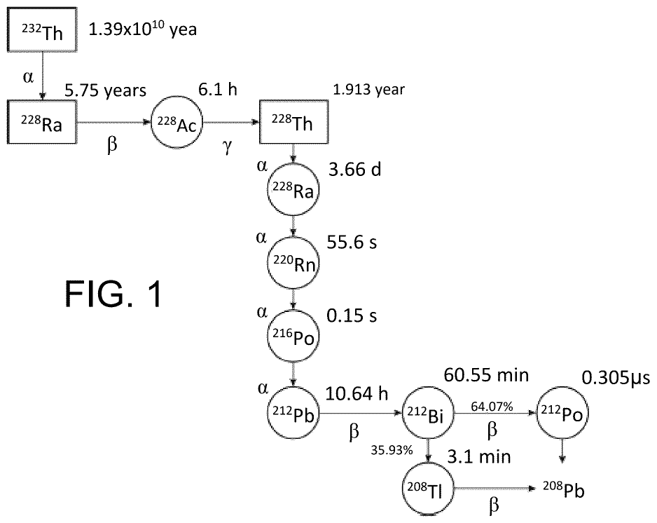

FIG. 2 schematically represents a first implementation mode of the method of the invention.

FIG. 3 schematically represents a second implementation mode of the method of the invention.

### DETAILED DISCLOSURE OF PARTICULAR IMPLEMENTATION MODES

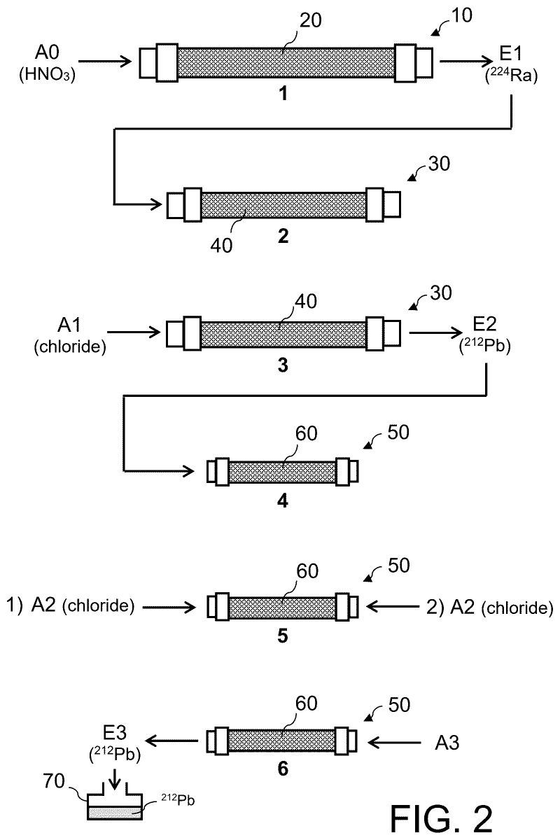

Reference is made to FIG. 2 which schematically illustrates the different steps, noted 1 to 6, of a first implementation mode of the method of the invention, in which a single Th-228/Ra-224 generator and a single Ra-224/Pb-212 generator are used.

In this implementation mode, the starting point of the method is therefore represented by a Th-228/Ra-224 generator, noted 10 in FIG. 2. This generator comprises a chromatography column whose stationary phase, noted 20, consists of DGA Normal (Triskem International) resin particles and to which thorium-228 is attached.

As thorium-228 present in the generator 10 has been allowed to decay to produce radium-224, the method comprises the following steps:
1. eluting thus produced radium-224 from the stationary phase 20 by means of an aqueous solution A0 of nitric acid to obtain an eluate E1 comprising radium-224;
2. preparing a Ra-224/Pb-212 generator, noted 30, by loading the eluate E1 into a chromatography column, the stationary phase 40 of which consists of AG™ MP-50 (Bio-Rad) resin particles, to attach radium-224 present in this eluate to the stationary phase 40;
3. radium-224 present in the generator 30 having been allowed to decay to produce lead-212, eluting this lead from the stationary phase 40 by means of an aqueous solution A1 of a chloride, alone or in mixture with very weakly concentrated hydrochloric acid, to obtain an eluate E2 comprising lead-212;
4. loading the eluate E2 into a chromatography column, noted 50, of which the stationary phase 60 consists of Resin PB (Triskem International) particles, to attach lead-212 present in this eluate to this stationary phase;
5. performing two successive washes of the stationary phase 60 to remove from the column 50, and in particular from the interstitial volume of this stationary phase, traces of radioisotopes other than lead-212 likely to be retained in this column in the previous step, each of these washes being carried out with an aqueous solution A2 of a chloride but in the opposite direction to each other;
6. eluting lead-212 from the stationary phase 60 by means of an aqueous solution A3 having a pH ranging from 5 to 9 and comprising one or more complexing agents and/or antioxidant agents to obtain an eluate E3 comprising purified lead 212 and collecting this eluate in a receptacle, noted 70, which may be of the beaker, flask, or similar type, as illustrated in FIG. 2, but which may also be a syringe connected to the tail of the column 50.

All these steps, which are detailed below, are carried out at room temperature, that is at a temperature of 20° C. to 25° C.

Furthermore, all solutions used are preferably Optima™ grade or prepared from Optima™ grade or Trace Metals grade reagents.

Unless otherwise indicated, the aqueous solutions that are circulated in columns 10, 30 and 50 are circulated from the column head to the column tail.

### *Step 1:

As previously indicated, this step consists in eluting, from the stationary phase 20 of the generator 10, radium-224 that has been produced by radioactive decay of thorium-228 attached to this stationary phase.

This generator comprises a chromatography column which has, for example, a bed volume, or BV, ranging from 5 mL to 100 mL and which is filled with DGA Normal resin particles, for example in an amount ranging from 2 g to 40 g of particles according to the BV of the column.

This type of resin retains thorium, regardless of the isotope thereof, but does not retain radium, regardless of the isotope thereof.

Eluting radium-224 is carried out by circulating in the generator 10 several BVs of aqueous solution A0—which comprises from 0.5 mol/L to 0.75 mol/L and, preferably, 0.5 mol/L nitric acid—at a flow rate which is, for example, 0.25 BV/min.

Thus, eluate E1 is obtained.

### *Step 2:

The generator 30 is prepared using a chromatography column of smaller dimensions than those of the chromatography column of the generator 10 and which has, for example, a BV ranging from 0.5 mL to 10 mL, and which is filled with AG™ MP-50 resin particles, for example in an amount ranging from 300 mg to 5 g of particles according to the BV of this column, and circulating eluate E1 in this column.

In chloride media, the AG™ MP-50 resin retains radium, regardless of the isotope thereof, but does not retain lead, regardless of the isotope thereof.

Loading the eluate E1 into the generator 30 is carried out at a flow rate which is, for example, 4 BV/min.

### *Step 3:

As previously indicated, this step consists, after a period of time during which the generator 30 has been allowed to produce lead-212 by radioactive decay of radium-224 present in this generator, in eluting thus produced lead-212 from the stationary phase 40.

To do this, several BVs of aqueous solution A1 are circulated in the generator 30, for example at a flow rate of 4 BV/min, this aqueous solution comprising from 0.8 mol/L to 1.6 mol/L chloride, advantageously magnesium chloride, and optionally hydrochloric acid, this acid, if present, has a concentration of at most 200 mmol/L, preferably at most 50 mmol/L and, still better, of 1 mmol/L.

Thus, eluate E2 is obtained.

### *Step 4:

Since lead-212 present in eluate E2 does not yet meet the radiological purity criterion required for medical use, steps 4, 5 and 6 aim to purify lead-212 with respect to its ascendants, and in particular with respect to radium-224, by means of a chromatography column filled with Resin PB.

The chromatography column 50 used for this purpose, of smaller dimensions than the chromatography column of the generator 30, has, for example, a BV ranging from 0.1 mL to 1 mL and is filled with 35 mg to 450 mg of Resin PB particles.

Loading eluate E2 into the column 50 is carried out by circulating this eluate in the column 50 at a flow rate which is, for example, 4 BV/min.

### *Step 5:

The two successive washes provided in this step are carried out with the same aqueous solution A2 but in the opposite direction to each other.

This aqueous solution comprises from 25 mmol/L to 75 mmol/L of the same chloride as that present in the aqueous solution A1 used in step 3 hereinabove, for example 50 mmol/L magnesium chloride.

The first wash is carried out by circulating in the column 50, from the column head to the column tail, several BVs of aqueous solution A2, for example at a flow rate of 6 BV/min, while the second wash is carried out by circulating in this column, preferably, the same number of BVs of aqueous solution A2 and at the same flow rate but from the column tail to the column head.

### *Step 6:

Eluting lead-212 from the stationary phase 60 is carried out by circulating in the column 50, from the column tail to the column head, several BVs of aqueous solution A3, which has a pH between 5 and 9 and comprises one or more complexing agents and/or antioxidant agents.

The aqueous solution A3 is, for example, an aqueous solution with a pH of 6.5 and comprising 0.4 mol/L ammonium acetate and 75 mmol/L citric acid.

It is circulated in the column 50 at a flow rate, for example, of 6 BV/min.

Eluate E3 thus obtained at the column head is collected in the container 70 and lead-212 present in this eluate has a radiological purity with respect to its ascendants allowing it to be used for medical purposes.

As previously indicated, the method as just described may also be implemented using n Th-228/Ra-224 generators, n being for example equal to 3, disposed in parallel or connected in series, as well as with m Ra-224/Pb-212 generators, m being for example equal to 2, also disposed in parallel or connected in series.

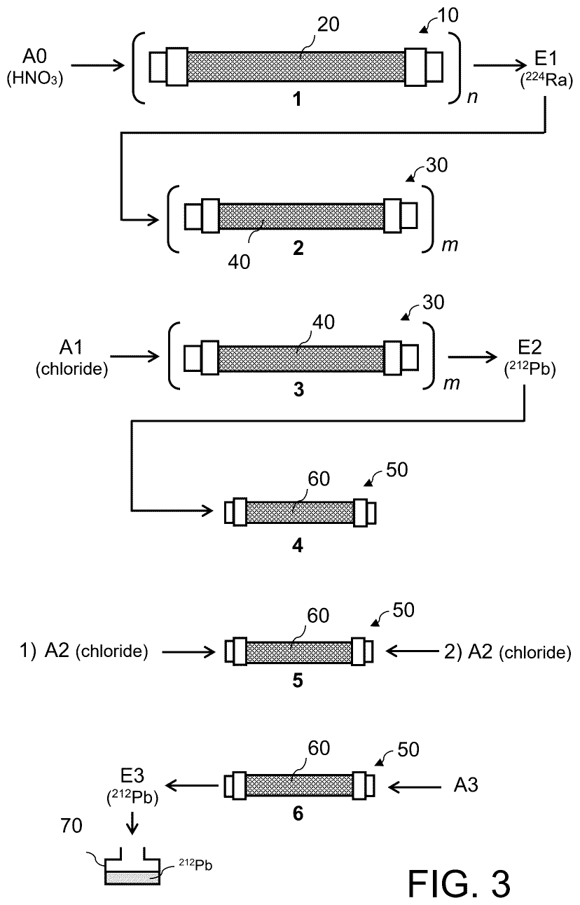

Thus, for example, as illustrated in FIG. 3 which illustrates an implementation mode of the method in which n Th-228/Ra-224 generators connected in series and m Ra-224/Pb-212 generators connected in series are used, this implementation mode differs from that represented in FIG. 2 only in that:
since thorium-228 has been allowed to decay in the n generators 10 to produce radium-224, step 1 is carried out by circulating the aqueous solution A0 of nitric acid successively in these n generators 10 or, in other words, by introducing this solution into the first of the n generators 10 and circulating it in the next n−1 generators 10, which results in obtaining a single eluate E1 comprising radium-224 upon leaving the n^{th} generator 10;
step 2 is carried out by circulating thus obtained eluate E1 successively in the m generators 30 or, in other words, by introducing this eluate into the first of the m generators 30 and circulating it in the next m−1 generators 30; and
since radium-224 has been allowed to decay in the m generators 30 to produce lead-212, step 3 is carried out by circulating the aqueous solution A1 successively in these m generators 30 or, in other words, by introducing this solution into the first of the m generators 30 and circulating it in the next m−1 generators 30, which results in obtaining a single eluate E2 comprising unpurified lead-212 upon leaving the m^{th} generator 30.

### Example 1—MgCl₂ 1 M/CLG BV 1.25 mL/CLL BV 0.173 mL

Steps 1 to 6 of the first implementation mode described above were carried out using:
*Step 1: 30 mL of an aqueous solution A0 comprising 0.5 mol/L nitric acid, at a flow rate of 5 mL/min, to elute radium-224 from the stationary phase of a generator 10 with a BV equal to 9.8 mL and containing 3.7 g of DGA resin;
*Step 2: a column 30 with a BV equal to 1.25 mL, containing 0.65 g of AG™ MP-50 resin and loaded, at a flow rate of 5 mL/min, with 25.4 mL of the eluate E1 obtained in the previous step, this eluate comprising 167 MBq of radium-224;
*Step 3: after 20 hours during which the radium-224 was allowed to produce lead-212, 10 mL of an aqueous solution A1 comprising 1 mol/L of MgCl₂, at a flow rate of 0.5 mL/min;
*Step 4: a column 50 with a BV equal to 0.173 mL (previously washed with 1 mL of an aqueous solution of MgCl₂ at 1.0 mol/L, at a flow rate of 0.5 mL/min), containing 73±5 mg of PB resin and loaded with the eluate E2 obtained in step 3, at a flow rate of 0.5 mL/min;
*Step 5: 2×1.5 mL of an aqueous solution A2 comprising 1.0 mol/L MgCl₂, at a flow rate of 0.5 mL/min;
*Step 6: 1.6 mL of an aqueous solution A3 comprising 0.4 mol/L ammonium acetate and 75 mmol/L citric acid, at a flow rate of 0.5 mL/min.

An aqueous solution containing 96.8 MBq of lead-212 was thus obtained.

This lead-212 had a radiological purity of more than 99.999% with respect to radium-224, as radium-224 was not detected after one week.

### Example 2—MgCl₂ 1 M/CLG BV=5.0 mL/CLL BV=0.63 mL

Steps 1 to 6 of the first implementation mode described above were implemented using:
*Step 1: 30 mL of an aqueous solution A0 comprising 0.5 mol/L nitric acid, at a flow rate of 5 mL/min, to elute radium-224 from the stationary phase of a generator 10 with a BV equal to 9.8 mL and containing 3.7 g of DGA resin;
*Step 2: a column 30 with a BV of 5 mL, containing 2.43 g of AG™ MP-50 resin, which was loaded at a flow rate of 5 mL/min with 37.8 mL of the eluate E1 obtained in the previous step, this eluate comprising 116 MBq of radium-224;
*Step 3: after 23 hours during which the radium-224 was allowed to produce lead-212, 30 mL of an aqueous solution A1 comprising 1 mol/L of MgCl₂, at a flow rate of 3.5 mL/min;
*Step 4: a column 50 with a BV equal to 0.63 mL (previously washed with 4 mL of an aqueous solution of MgCl₂ at 1.0 mol/L, at a flow rate of 3.5 mL/min), containing 235±15 mg of PB resin and loaded with the eluate E2 obtained in step 3, at a flow rate of 3.5 mL/min;
*Step 5: 2 times 3 mL of an aqueous solution A2 comprising 1.0 mol/L MgCl₂, at a flow rate of 3.5 mL/min;
*Step 6: 6 mL of an aqueous solution A3 comprising 0.4 mol/L ammonium acetate and 75 mmol/L citric acid, at a flow rate of 3.5 mL/min.

An aqueous solution containing 75.7 MBq of lead-212 was thus obtained.

This lead-212 had a radiological purity of more than 99.999% with respect to radium-224, as radium-224 was not detected after one week.

### Example 3—MgCl₂ 1 M+1 mM HCl/CLG BV=1.25 mL/CLL BV=0.173 mL

Steps 3 to 6 of the first implementation mode described above were implemented following Example 1 using:
*Step 3: after 24 hours during which the radium-224 was allowed to produce lead-212, 10 mL of an aqueous solution A1 comprising 1 mol/L of MgCl₂ and 1 mmol/L of HCl, at a flow rate of 0.5 mL/min;
*Step 4: a column 50 with a BV equal to 0.173 mL (previously washed with 1 mL of an aqueous solution of 1.0 mol/L MgCl₂ and 1 mmol/L HCl, at a flow rate of 0.5 mL/min), containing 73±5 mg of PB resin and loaded with the E2 eluate obtained in step 3, at a flow rate of 0.5 mL/min;
*Step 5: 2×1.5 mL of an aqueous solution A2 comprising 1.0 mol/L MgCl₂ and 1 mmol/L HCl, at a flow rate of 0.5 mL/min;
*Step 6: 1.6 mL of an aqueous solution A3 comprising 0.4 mol/L ammonium acetate and 75 mmol/L citric acid, at a flow rate of 0.5 mL/min.

An aqueous solution containing 77.5 MBq of lead-212 was thus obtained.

This lead-212 had a radiological purity of more than 99.999% with respect to radium-224, as radium-224 was not detected after one week.

### Example 4—MgCl₂ 1 M+1 mM HCl/CLG BV=5.0 mL/CLL BV=0.63 mL

Steps 3 to 6 of the first implementation mode described above were implemented following Example 2 using:
*Step 3: after 71 hours during which the radium-224 was allowed to produce lead-212, 30 mL of an aqueous solution A1 comprising 1 mol/L of MgCl₂ and 1 mmol/L of HCl, at a flow rate of 3.5 mL/min;
*Step 4: a column 50 with a BV equal to 0.63 mL (previously washed with 4 mL of an aqueous solution of 1.0 mol/L MgCl₂ and 1 mmol/L HCl, at a flow rate of 3.5 mL/min), containing 235±15 mg of PB resin and loaded with the eluate E2 obtained in step 3, at a flow rate of 3.5 mL/min;
*Step 5: 2×3 mL of an aqueous solution A2 comprising 1.0 mol/L MgCl₂ and 1 mmol/L HCl, at a flow rate of 3.5 mL/min;
*Step 6: 6 mL of an aqueous solution A3 comprising 0.4 mol/L ammonium acetate and 75 mmol/L citric acid, at a flow rate of 3.5 mL/min.

An aqueous solution containing 51.5 MBq of lead-212 was thus obtained.

This lead-212 had a radiological purity of more than 99.999% with respect to radium-224, as radium-224 was not detected after one week.

In conclusion from examples 1 to 4, the yields of Pb-212 collected relative to lead produced by decay were 88%, 77%, 95% and 85% respectively. Purities are greater than 99.999%, with purity calculations taking into account detection limit values, as Ra-224 was not detected.

### REFERENCES CITED

[1] WO-A-2013/174949
[2] WO-A-2017/093069

1. A method for producing a medical grade aqueous lead-212 solution, which comprises at least the steps of:
a) producing lead-212 by radioactive decay of radium-224 present in at least one first chromatography column containing a first stationary phase to which radium-224 is attached;
b) eluting thus produced lead-212 from the first stationary phase to obtain an eluate comprising unpurified lead-212;
c) loading the eluate thus obtained into a second chromatography column containing a second stationary phase to attach lead-212 present in the eluate to the second stationary phase;
d) performing wash(es) of the second stationary phase to remove radioactive impurities likely to be retained by the second stationary phase without removing lead-212; and then
e) eluting lead-212 from the second stationary phase, whereby medical grade lead-212 is obtained in aqueous solution;
and wherein:
step b) comprises circulating in said at least one first chromatography column an aqueous solution A1 comprising from 0.8 mol/L to 1.6 mol/L chloride, alone or in mixture with at most 200 mmol/L hydrochloric acid; and
step d) comprises circulating in the second chromatography column an aqueous solution A2 comprising from 0.01 mol/L to 1 mol/L chloride.

2. The method according to claim 1, wherein the chloride present in the aqueous solutions A1 and A2 is an alkali metal chloride, an alkaline earth metal chloride, or ammonium chloride.

3. The method according to claim 2, wherein the chloride is magnesium chloride.

4. The method according to claim 1, wherein the aqueous solution A1 comprises from 0.8 mol/L to 1.2 mol/L, alone or in mixture with at most 50 mmol/L hydrochloric acid.

5. The method according to claim 1, wherein the aqueous solution A1 comprises 1.0 mol/L magnesium chloride, alone or in mixture with at most 50 mmol/L hydrochloric acid.

6. The method according to claim 1, wherein the aqueous solution A2 comprises from 0.1 mol/L to 1 mol/L magnesium chloride.

7. The method according to claim 1, wherein the aqueous solution A2 comprises 1 mol/L magnesium chloride.

8. The method according to claim 1, wherein the second chromatography column has a column head and a column tail and step d) comprises circulating a first volume of aqueous solution A2 from the column head to the column tail, and then circulating a second volume of aqueous solution A2 from the column tail to the column head.

9. The method according to claim 1, wherein step e) comprises circulating in the second chromatography column an aqueous solution A3 with a pH between 5 and 9 and comprising one or more complexing agents and/or antioxidant agents.

10. The method according to claim 9, wherein the aqueous solution A3 comprises ammonium acetate and citric acid and/or DOTAM.

11. The method according to claim 10, wherein the aqueous solution A3 comprises 0.4 mol/L ammonium acetate and 75 mmol/L citric acid.

12. The method according to claim 9, wherein the second chromatography column has a column head and a column tail and the aqueous solution A3 circulates in the second chromatography column from the column tail to the column head.

13. The method according to claim 1, wherein m first chromatography columns disposed in parallel and each containing a first stationary phase to which radium-224 is attached, m being an integer at least equal to 2, are used, and:
steps a) and b) are performed in each of the first m chromatography columns, whereby m eluates comprising unpurified lead-212 which are collected separately or together to form a mixture of the m eluates are obtained;
step c) is performed by loading the m eluates or the mixture of the m eluates thus obtained into the second chromatography column containing the second stationary phase;
step d) of washing the second stationary phase is performed; and
step e) of eluting lead-212 from the second stationary phase is performed.

14. The method according to claim 1, wherein m first chromatography columns connected in series and each containing a first stationary phase to which radium-224 is attached, m being an integer at least equal to 2, are used, and:
steps a) and b) are performed in each of the first m chromatography columns, step b) being carried out by circulating the aqueous solution A1 successively in the first m chromatography columns, whereby an eluate comprising unpurified lead-212 is obtained when leaving the m^{th} of the m chromatography columns;
step c) is performed by loading the eluate thus obtained into the second chromatography column containing the second stationary phase;
step d) of washing the second stationary phase is performed; and
step e) of eluting lead-212 from the second stationary phase is performed.

15. The method according to claim 14, wherein m is 2 or 3.

16. The method according to claim 1, which further comprises, prior to step a), the steps of:
i) producing radium-224 by radioactive decay of thorium-228 in at least one third chromatography column comprising a third stationary phase to which thorium-228 is attached; and
ii) eluting thus produced radium-224 from the third stationary phase to obtain an eluate comprising radium-224, eluting comprising circulating in said at least one third chromatography column an aqueous solution A0 comprising from 0.4 mol/L to 1 mol/L nitric acid; and then
iii) loading the eluate thus obtained into said at least one first chromatography column to attach radium-224 present in the eluate to the first stationary phase.

17. The method according to claim 16, wherein the aqueous solution A0 comprises 0.5 mol/L nitric acid.

18. The method according to claim 16, wherein n third chromatography columns disposed in parallel and each containing a third stationary phase to which thorium-228 is attached, n being an integer at least equal to 2, are used, and:
steps i) and ii) are performed in each of the n third chromatography columns, whereby n eluates comprising radium-224 which are collected separately or together to form a mixture of the n eluates are obtained; and
step iii) is performed by loading the n eluates or the mixture of the n eluates thus obtained into said at least one first chromatography column.

19. The method according to claim 16, wherein n third chromatography columns connected in series and each containing a third stationary phase to which thorium-228 is attached, n being an integer at least equal to 2, are used, and:
steps i) and ii) are performed in each of the n third chromatography columns, step ii) being performed by circulating the aqueous solution A0 successively in the n third chromatography columns, whereby an eluate comprising radium-224 is obtained when leaving the n^{th} of the n third chromatography columns; and
step iii) is performed by loading the eluate thus obtained into said at least one first chromatography column.

20. The method according to claim 19, wherein n is 2 or 3.
